(19) United States
(12) Patent Application Publication
(10) Pub. No.: US 2012/0103345 A1
(43) Pub. Date: May 3, 2012

(54) Sleeping Aid

(76) Inventor: Maxwell Harold Gay, Lower Hutt (NZ)

(21) Appl. No.: 13/267,682

(22) Filed: Oct. 6, 2011

(30) Foreign Application Priority Data
Nov. 2, 2010 (NZ) 588969

(51) Int. Cl. A61F 5/56

(52) U.S. Cl. 128/848

(57) ABSTRACT

A sleeping aid having an upper moulding, a lower moulding and a bridge; the sleeping aid formed so that it can be placed in the mouth of a human user with a series of the user's upper teeth snugly in the upper moulding, a series of the user's lower teeth snugly in the lower moulding, the user's lower jaw held forward of its natural resting position by the mouldings, and the bridge extending across the user's tongue to restrict the tongue from rearward movement; the sleeping aid formed such that when it is worn in this way the forward position of the lower jaw and the position of the bridge substantially inhibits snoring and/or sleep apnoea.

### CROSS-REFERENCE TO RELATED APPLICATIONS

This application claims the benefit of New Zealand Provisional Application No. 588969, filed Nov. 2, 2010. The entire disclosure of the prior application is hereby incorporated by reference.

### FIELD OF INVENTION

This invention relates to a sleeping aid. A preferred form of the invention relates to a sleeping aid which alleviates snoring and/or sleep apnoea

### BACKGROUND

It is known for people to snore loudly when sleeping, or to suffer from sleep apnoea. It has been found that causing the lower jaw of a person to be held slightly forward of its normal position reduces the tendency for snoring or symptoms of sleep apnoea. A device for achieving this is described in patent specification WO 94/23674 in the name of Jacobsen. However for some people the Jacobsen device does not provide a complete solution. It is an object of a preferred form of the invention to go at some way towards providing an alternative sleeping aid which proves effective for at least some people.

### SUMMARY OF THE INVENTION

According to one aspect of the invention there is provided a sleeping aid having an upper moulding, a lower moulding and a bridge;

the sleeping aid formed so that it can be placed in the mouth of a human user with a series of the user's upper teeth snugly in the upper moulding, a series of the user's lower teeth snugly in the lower moulding, the user's lower jaw held forward of its natural resting position by the mouldings, and the bridge extending across the user's tongue to restrict the tongue from rearward movement;

the sleeping aid formed such that when it is worn in this way the forward position of the lower jaw and the position of the bridge substantially inhibits snoring and/or sleep apnoea.

Preferably the mouldings have been shaped to match the mouth of an individual user.

Preferably the bridge is formed so that when the sleeping aid is worn as above the bridge extends laterally with respect to the rest of the sleeping aid adjacent to the user's rearmost teeth.

Preferably the sleeping aid is formed to hold the user's lower jaw 3 mm to 8 mm forward of its natural resting position.

Preferably the sleeping aid has a canopy shaped to match and lay against the roof of the user's mouth.

Preferably the sleeping aid is formed from a soft plastic material.

Preferably the sleeping aid has a breathing opening between the mouldings.

Preferably the bridge is positioned so that it does not contact the tongue when the tongue is in a normal resting position sufficiently clear of the user's breathing passage to avoid snoring and/or symptoms of sleep apnoea.

### BRIEF DESCRIPTION OF THE DRAWINGS

Some preferred forms of the invention with now be described by way of example and with reference to the accompanying images, of which:

FIG. 1 is an isometric top view of a sleeping aid;

FIG. 2 is an isometric view of the underside of the sleeping aid;

FIG. 3 is an isometric side view of the sleeping aid; and

FIG. 4 is an isometric front view of the sleeping aid.

### DETAILED DESCRIPTION

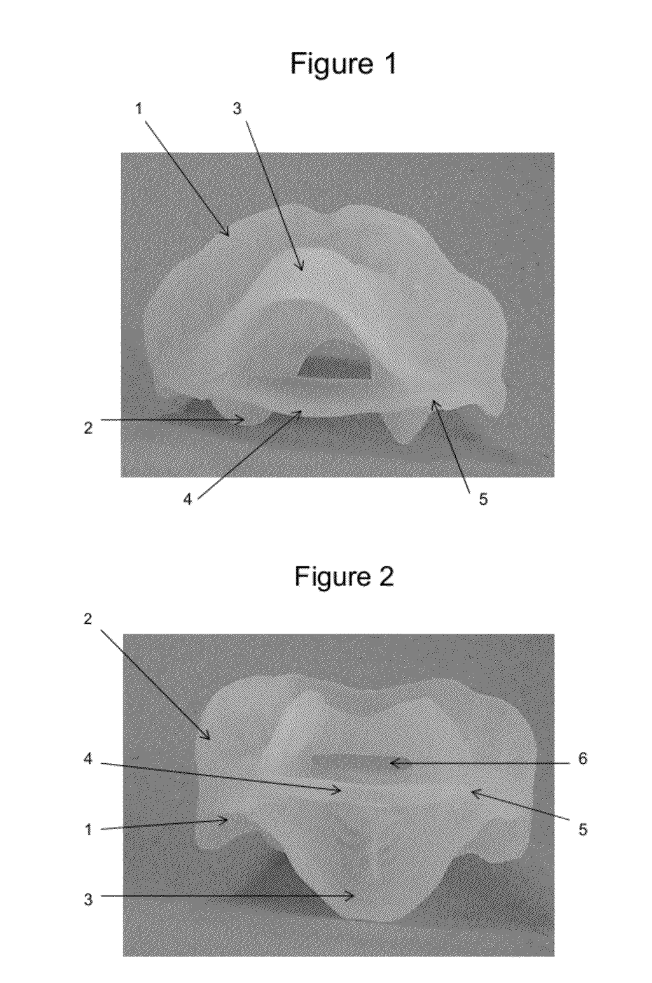

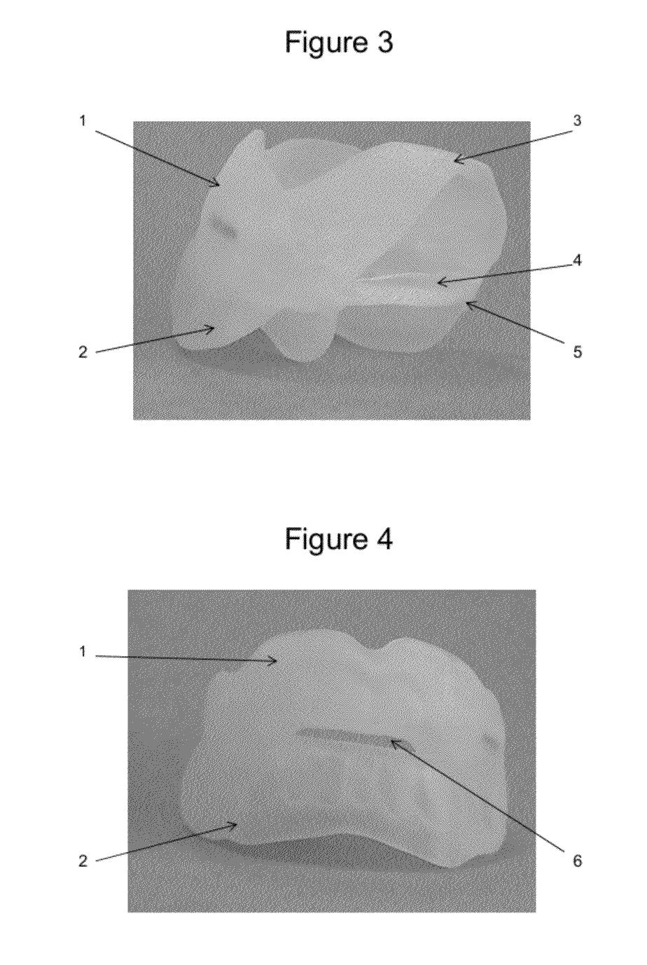

The sleeping aid is shaped from soft plastic to fit the mouth of a human user prone to snoring or sleep apnoea. In a general sense it resembles and is worn in a similar manner to an athletic double mouth guard of the sort used in contact sports such as boxing. The sleeping aid has an upper moulding 1 and a lower moulding 2 shaped to match the upper and lower rows of the user's teeth respectively. The upper moulding 1 merges into a canopy 3 shaped to match the roof of the user's mouth. As shown in FIGS. 1, 2 and 3, a bridge 4 extends from one side of the sleeping aid to the other at about the level 5 where the mouldings are joined. As most clearly seen in FIGS. 2 and 4, a breathing aperture 6 is situated in the front of the sleeping aid between the mouldings 1, 2.

A common cause of snoring or sleep apnoea is that, while the person concerned is sleeping, their tongue moves into a position where it obstructs the normal breathing passage at the back of the throat. To address this the sleeping is placed into the user's mouth so that the upper row of teeth is within the upper moulding, the lower row of teeth is within the lower moulding, the canopy 3 is against the roof or the mouth and the bridge 4 lays across the top of the tongue about as far back into the mouth as the most rearward teeth.

The sleeping aid is formed so that the lower moulding 2 holds the user's lower jaw slightly further forward, for example by 3-8 mm, of its natural resting position. In some versions the sleeping aid may be formed so that it holds the lower jaw forward by an amount which is 60% to 80% of the distance between the lower jaw when in its natural resting position and when in its maximum possible forward position. In this regard the upper and lower mouldings are joined so as to hold them in a fixed position with respect to one another. Holding the lower jaw forward serves to keep the tongue away from the breathing passage at the back of the throat so that it is sufficiently clear to avoid snoring or symptoms of sleep apnoea.

With some people the tongue is prone to moving back, for example in a flopping action, to cause an air passage obstruction even when the lower jaw is held in a forward position. However the bridge 4 addresses this by providing a barrier against backwards movement of the tongue. Preferably the bridge is set so that when a user is sleeping it is just off the tongue, and only makes contact with the tongue if it moves towards what would be an air passage obstructing position.

The sleeping aid may be formed in any suitable way and from any suitable material. For best results it should be custom made to match the mouth of its intended user. By way of example, an impression is taken of the user's upper teeth and associated gums and roof of the mouth. An impression is also taken of the lower teeth and associated gums. The impressions are used to make hard models using dental stone. These are in turn used for shaping the upper and lower mouldings 1, 2 by applying heat softened plastic material over the models. The plastic is preferably 2-3 mm thick. The mouldings are then cooled and trimmed to remove undesirable excess plastic.

A bite register is taken with the user's teeth in the forward position described above. A register may also, or alternatively, be taken for the forward position just before the teeth make biting contact. These registers are used to determine the position of the upper and lower mouldings relative to one another. The mouldings 1, 2 are then fused using heat and, if necessary, additional soft plastic. The breathing aperture 6 may be formed last.

While some embodiments of the invention have been described by way of example it should be appreciated that modifications and improvements can occur without departing from the scope of the following claims.

1. A sleeping aid having an upper moulding, a lower moulding and a bridge;
the sleeping aid formed so that it can be placed in the mouth of a human user with a series of the user's upper teeth snugly in the upper moulding, a series of the user's lower teeth snugly in the lower moulding, the user's lower jaw held forward of its natural resting position by the mouldings, and the bridge extending across the user's tongue to restrict the tongue from rearward movement;
the sleeping aid formed such that when it is worn in this way the forward position of the lower jaw and the position of the bridge substantially inhibits snoring and/or sleep apnoea.

2. A sleeping aid according to claim 1, wherein the mouldings have been shaped to match the mouth of an individual user.

3. A sleeping aid according to claim 1, wherein the bridge is formed so that when the sleeping aid is worn as above the bridge extends laterally with respect to the rest of the sleeping aid adjacent to the user's rearmost teeth.

4. A sleeping aid according to claim, formed to hold the user's lower jaw 3 mm to 8 mm forward of its natural resting position.

5. A sleeping aid according to claim 1, having a canopy shaped to match and lay against the roof of the user's mouth.

6. A sleeping aid according to claim 1, formed from a soft plastic material.

7. A sleeping aid according to claim 1, having a breathing opening between the mouldings.

8. A sleeping aid according to claim 1, wherein the bridge is positioned so that it does not contact the tongue when the tongue is in a normal resting position sufficiently clear of the user's breathing passage to avoid snoring and/or symptoms of sleep apnoea.

9. A sleeping aid having an upper moulding, a lower moulding, a breathing opening between the mouldings and a bridge;
the upper and lower mouldings having shaped to match the mouth of an individual user;
the sleeping aid formed so that it can be placed in the mouth of a human user with a series of the user's upper teeth snugly in the upper moulding, a series of the user's lower teeth snugly in the lower moulding, the user's lower jaw held forward of its natural resting position by the mouldings, and the bridge extending laterally across the user's tongue to restrict the tongue from rearward movement;
the sleeping aid formed such that when it is worn in this way the forward position of the lower jaw and the position of the bridge substantially inhibits snoring and/or sleep apnoea

10. A sleeping aid according to claim 10, having a canopy shaped to match and lay against the user's mouth when the sleeping aid is in normal use.

11. A sleeping aid according to claim 9, formed to hold the user's jaw 3 mm to 8 mm forward of its natural resting position.
